(19) United States
(12) Patent Application Publication
(10) Pub. No.: US 2014/0107396 A1
(43) Pub. Date: Apr. 17, 2014

(54) SYSTEMS AND METHODS FOR DELIVERING A THERAPEUTIC AGENT

(71) Applicant: Lutz Freitag, Hemer (DE)

(72) Inventor: Lutz Freitag, Hemer (DE)

(73) Assignee: PulmonX Corporation, Redwood City, CA (US)

(21) Appl. No.: 13/830,642

(22) Filed: Mar. 14, 2013

Related U.S. Application Data
(63) Continuation-in-part of application No. 10/241,733, filed on Sep. 10, 2002.
Application No. 10/241,733 is a continuation-in-part of application No. 12/474,169, filed on May 28, 2009.
(60) Provisional application No. 61/615,029, filed on Mar. 23, 2012.

(51) Int. Cl. A61N 5/10; A61B 17/12

(52) U.S. Cl. CPC A61N 5/10; A61N 5/1001; A61B 17/12104
USPC 600/3; 600/1

(57) ABSTRACT

Devices, systems, and methods for delivering a therapeutic agent to a treatment region in the body are disclosed. By determining a treatment region in the body, reducing the volume of the treatment region to create a target region by using one or more flow control elements and delivering at least one therapeutic agent to the target region, improved treatment may be achieved.

### CROSS-REFERENCE TO RELATED APPLICATIONS

This application is a continuation-in-part of U.S. patent application Ser. No. 10/241,733 filed on Sep. 10, 2002 entitled “Method and Apparatus for Endobronchial Diagnosis” by Kotmel et al, the full disclosure of which is also incorporated herein by reference; this application is also a continuation-in-part of U.S. patent application Ser. No. 12/474,169 entitled “Methods and Systems for Assessing Lung Function and Delivering Therapeutic Agents” by Aljuri et al. filed on May 28, 2009, the full disclosure is hereby incorporated herein by reference; this application also claims the benefit under 37 C.F.R. Section 1.78 of U.S. Provisional Application No. 61/615,029 entitled “Systems and Methods for Delivering a Therapeutic Agent” by Freitag filed on Mar. 23, 2012, the full disclosure of which is also incorporated herein by reference.

### BACKGROUND

Present disclosure relates generally to devices, systems, and methods for delivering a therapeutic agent to a treatment region in the body, more specifically, to a treatment region in the lung.

Lung cancer is characterized by the uncontrolled cell growth in the lung. In general, there are two main categories of lung cancer: non-small cell lung cancer and small cell lung cancer. Non-small cell lung cancer may be treated using surgical resection, radiation, chemotherapy or any combinations thereof. Typical surgical resection for treating lung cancer includes pneumonectomy, lobectomy, wedge resection, and segmental resection. Although surgical resection can be an effective treatment option, it is not a viable option for many patients due to the location of the tumor, whether the cancer has spread to both lungs and other structures in the chest, the lymph nodes, or other organs. Surgical resection may also result in complications with anesthesia or infection, and can result in extended recovery periods.

Radiation therapy involves the use of high-energy rays or particles to kill cancer cells. Radiation therapy has become a significant and highly successful process particularly for treating localized cancers including lung cancer. Radiation therapy is particularly useful for treating centrally located tumors and/or small cell tumors that cannot be removed surgically. Radiation therapy can be used as a curative treatment or as a palliative treatment when a cure is not possible. Additionally, surgery and chemotherapy can be used in combination with radiation therapy.

There are two commonly practiced forms of radiation therapy—external beam radiation therapy and internal radiation therapy also known as brachytherapy. In general, internal radiation therapy or brachytherapy is used to shrink tumors and to relieve symptoms caused by lung cancer in an airway. This procedure is usually performed by placing a small amount of radioactive material, often in the form of pellets or seeds, either directly into the cancer or into the airway next to the cancer. External beam radiation therapy involves delivering radiation energy to a location in the body for a period of time. The typical procedure of external beam radiation therapy includes (a) a planning process to determine the parameters of the radiation, (b) a target process where the desired targeted location where the radiation beam will be delivered to the body is determined, (c) radiation sessions where the radiation beam is delivered to the targeted location to irradiate the cancer, and (d) qualification processes to assess the efficacy of the radiation sessions. Many radiation therapy procedures typically have multiple radiation sessions over a treatment period.

To further improve the radiation therapy treatment, it would be desirable to increase the radiation dose because higher doses are more effective at destroying most cancers. Increasing the radiation dose, however, also increases the potential for complications to surrounding healthy tissues. The efficacy of radiation therapy accordingly depends on both the total dose of radiation delivered to the tumor and the dose of radiation delivered to normal tissue adjacent to the tumor. To protect the normal tissue adjacent to the tumor, the radiation should be prescribed to a tight treatment margin around the target to avoid irradiating healthy tissue. In particular, it would be desirable to decrease the targeted location where the radiation will be delivered such that the higher dose of radiation may be prescribed while decreasing irradiation of healthy tissue.

### SUMMARY

Devices, systems, and methods are provided for treating a treatment region in the body by delivering one or more therapeutic agents to the treatment region.

In one aspect, methods are provided for treating a body region by determining a treatment region, reducing the volume of the treatment region to create a target region, and delivering at least one therapeutic agent to the target region. In such aspect, reducing the volume of the treatment region is achieved by deploying one or more flow control elements to reduce fluid flow into the treatment region.

In another aspect, methods are provided for treating a lung region by delivering one or more flow control elements to a lung region, deploying the flow control elements to at least partially collapse the lung region and thereafter delivering at least one therapeutic agent to the collapsed lung region, wherein the effect of the therapeutic agent is at least partially contained within the collapsed lung region.

This, and further aspects of the present embodiments are set forth herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 illustrates a flow diagram showing one exemplary method of delivering one or more therapeutic agents to the treatment region.

FIG. 2 illustrates one embodiment of the flow control element.

FIG. 3A illustrate the diagnosis of a patient before the treatment of using one embodiment of the present disclosure.

FIG. 3B illustrate the diagnosis of a patient after the treatment using one embodiment of the present disclosure.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

The present disclosure describes devices, systems, and methods of treating a body region using one or more therapeutic agents. Specifically, the embodiments of the present disclosure describe devices, systems, and methods for treating a body region, such as a lung region, where the lung region is at least partially collapsed to achieve volumetric reduction, and at least one therapeutic agent is delivered to the volumetrically reduced lung region.

Various medical indications require delivering one or more therapeutic agents to a body region. Often, a targeted delivery of the therapeutic agents to a specific body region is preferable such that the effect of the therapeutic agent is regionalized to maximize the treatment effect and to enable higher dosage prescription while minimizing the effect of the surrounding healthy tissue. However, due to anatomical limitations or other considerations, it may be difficult to target the delivery and/or the therapeutic effect of a therapeutic agent to the desirable body region. For example, lung cancer with lymphonodular metastasis with a large primarius in a peripheral location within a lung region can be a difficult target to achieve a high dosage radiation therapy. In order to improve the outcome of the patients and to prohibit long-term damage, the present devices, systems, and methods are configured to reduce the volume of a treatment region and deliver a therapeutic agent to the reduced treatment region to enhance therapeutic effect of the agent to the treatment region while reduce the effect on surrounding tissue.

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conforms to nomenclature for portions of the lungs that are known to those skilled in the art. However, it should be appreciated that the term “lung region” does not necessarily refer to a lung lobe or a lung segment, but can refer to some other defined division or portion of a human or non-human lung. Furthermore, as used herein, the term “lung segment” refers to a bronchopulmonary segment that is an anatomically distinct unit or compartment of the lung which is fed air by a tertiary bronchus and which oxygenates blood through a tertiary artery.

Although the embodiments described herein use the lung region as an exemplary body region, it is noted that the present disclosure is not limited to treating the lung region and it is contemplated that the present embodiments may be used in any air passageway or body lumen.

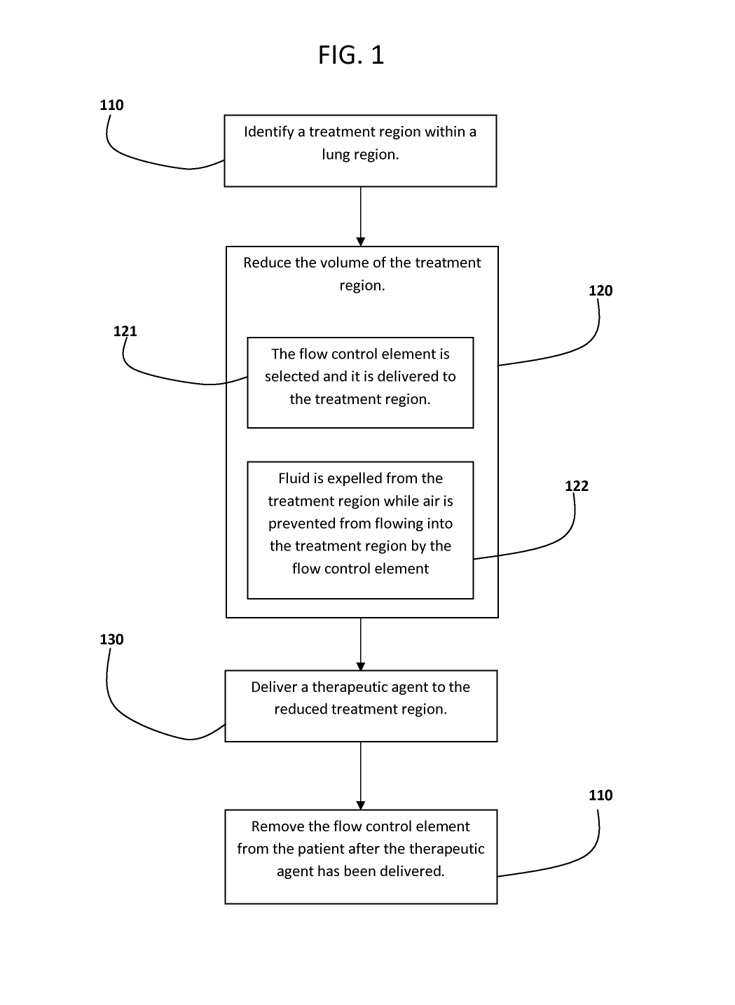

Referring now to FIG. 1, which is a flow diagram illustrating steps of one exemplary method of the present disclosure. At step 110, a treatment region within a lung region is identified. In one embodiment, the treatment region may be a lung region comprising one or more tumors. Such treatment region may be identified using CT scan, magnetic resonance tomography, sonography, or any other diagnostic techniques. In another embodiment, the treatment region may be a lung region comprising or affected by another indication and/or disease.

At step 120, the volume of the treatment region is reduced. In one embodiment, the volumetric reduction of the treatment region is caused by a partial collapse of the treatment region. In another embodiment, the volumetric reduction of the target portion is caused by a total collapse of the treatment region. In one embodiment, the partial or total collapse of the treatment portion of the lung is achieved by using at least one flow control element to reduce or substantially eliminate fluid flow into the treatment region.

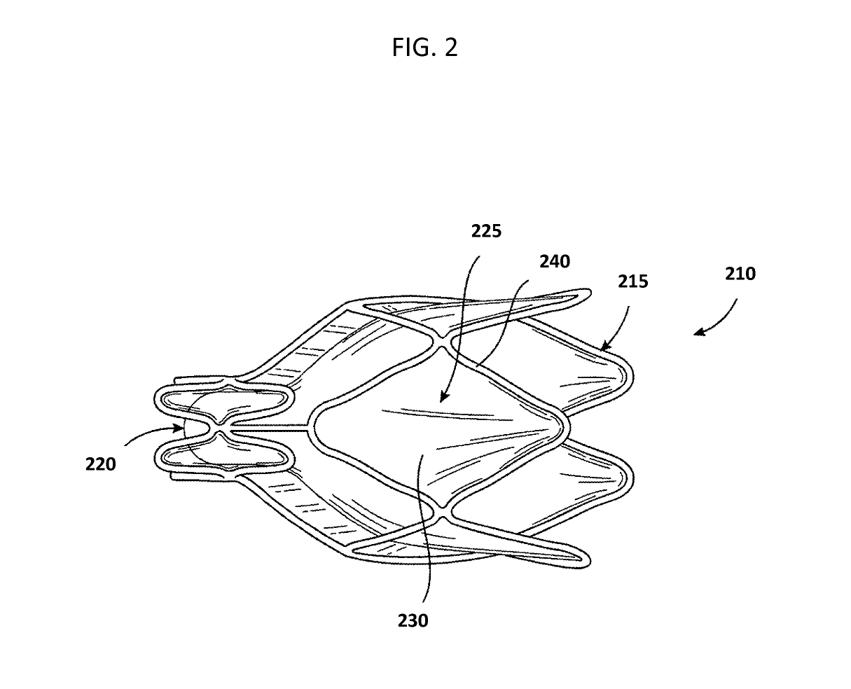

In one embodiment, the flow control element is capable of one-way flow, the sealing of a body passageway and/or pressure actuation. In one embodiment, the flow control element is an endobronchial valve as seen in FIG. 2. As seen in FIG. 2, the flow control element 210 includes a frame 215, a valve member 220 mounted in the frame, and a membrane 225. As seen in FIG. 2, the frame 215 comprises a plurality of interconnected struts 240. Although the embodiment of the flow control element shown in FIG. 2 is configured to be positioned within a bronchial passageway to regulate fluid flow through the bronchial passageway, it is envisioned that the embodiment may be modified to be operable within other body regions. The valve member 220 as seen in FIG. 2 is disposed within the valve protection portion of the frame 215. The valve member 220 can be configured to either permit fluid flow in two directions (i.e., proximal and distal directions), permit fluid flow in only one direction (proximal or distal direction), completely restrict fluid flow in any direction through the flow control element 210, or any combination of the above. The valve member 220 can be configured such that when fluid flow is permitted, it is only permitted above a certain pressure, referred to as the cracking pressure. The valve member 220 is desirably formed of an elastic, biocompatible material, such as silicone, although other materials can be used. Additional embodiments of the flow control element are disclosed in U.S. Pat. No. 5,954,766; U.S. Pat. No. 6,679,264; U.S. Pat. No. 6,941,950; and U.S. Pat. No. 7,798,147. The foregoing references are all incorporated by reference in their entirety and are all assigned to the assignee of the instant application.

Alternatively, the flow control element may be a device configured to reduce the rate of air exchange between the treatment region and the feeding airway or bronchus while allowing a reduced rate of air flow in both the inhalation or inspiratory direction and the exhalation or expiratory direction as described in the co-pending U.S. Pat. Ser. No. 11/682,986 which is incorporated by reference in its entirety and is assigned to the assignee of the instant application.

Furthermore, it is contemplated that the flow control element may be any device configured to restrict, limit, or block air flow into the treatment region, such as various configurations of plugs, valves, partial or complete occlusive devices, etc.

At sub-step 121, one of the above described embodiments of the flow control element is selected and it is delivered to the treatment region. In one embodiment, the flow control element is delivered to the treatment region in the lung using a delivery catheter that is advanced through the mouth, down through the trachea and through the main bronchus. Thereafter, the delivery catheter may be further advanced to an airway which feeds the treatment region. In one embodiment, the delivery catheter may be introduced through the main bronchus with or without the use of a bronchoscope or other introducing catheter. Additionally or alternatively, the delivery catheter may be introduced into the treatment region though a scope, such as a visualizing endotracheal tube, which his capable of advancing into the branching airways of the lung region. It is further contemplated that various elements may be used to assist the delivery catheter, for example, a balloon or cuff that is optionally disposed on the catheter may be used to immobilize or stabilize the delivery catheter. Once the delivery catheter has been placed in a desired position, the flow control element is deployed from the delivery catheter using a deployment means such as a pusher that can be advanced to eject the flow control element from the delivery catheter to the treatment region.

Thereafter, at sub-step 122, in an embodiment where the flow control element is a endobronchial valve, through normal breathing cycle, fluid such as air is expelled from the treatment region while air is prevented from flowing into the treatment region by the flow control element, such that partial or complete atelectasis of the treatment region is achieved. Alternatively, in an embodiment where the flow control element is a bi-directional flow restrictor, air flow into and out of the segment as the patient inhales and exhales will be restricted and partial or complete atelectasis is achieved over time. Alternatively and optionally, aspiration techniques may be used to facilitate partial or complete atelectasis.

Due to the shrinking of the atelectatic treatment region, the volume of the treatment region is now reduced. At step 130, a therapeutic agent is delivered to the reduced treatment region. In one embodiment, the therapeutic agent is radioactive energy delivered to the treatment region during external beam radiation therapy to treat the cancer in the treatment region. In such embodiment, external beam radiation therapy such as 2D radiation therapy, 3D conformal radiation therapy, Intensity Modulated Radiation Therapy (IMTR), stereotatic radiation therapy, proton beam therapy or the like may be employed. Typically, during the external beam radiation therapy, the oncologist first needs to determine the target region to aim the radioactive energy and calculate the dose of radiation Due to the volumetric reduction of the treatment region, a better approximation of the area of treatment region where the therapeutic agent is to be delivered may be ascertained with greater precision such that the target region may be reduced. Additionally and optionally, the dosage of radiation may be increased since the greater precision in aiming the radioactive energy due to the reduced treatment region allows more focused delivery with greater protection for the surrounding tissue.

Additionally, it is contemplated that the therapeutic agent may be any other substance suitable for the intended treatment objective. In one embodiment, the therapeutic agent may be a solid therapeutic agent such as a radioactive pellet or seed used in internal radiation therapy that is inserted into the reduced treatment region via bronchoscopy. Due to the reduced treatment region the radioactive seed may be inserted closer to the tumor with greater precision.

Alternatively, the therapeutic agent may be a flowable therapeutic agent such as anti-microbial agents such as adrenergic agents, antiviral agents, antibiotic agents or antibacterial agents, anthelmintic agents, anti-inflammatory agents, antineoplastic agents, antioxidant agents, biological reaction inhibitors, botulinum toxin agents, chemotherapy agents, diagnostic agents, gene therapy agents, hormonal agents, and/or mucolytic agents. Due to the reduced treatment region, the therapeutic agent may be delivered to the treatment site with greater precision. Additionally, the reduced treatment region and optionally in conjunction with the flow control element may aid in containing or confining the therapeutic agent within the treatment site such that the effect of the therapeutic agent is at least partially contained within the treatment region. Additionally, present devices, systems, and methods may be beneficial by inhibiting exhalation and/or mucociliary transport by isolating or confining the involved treatment portion to prevent disease dissemination.

In the different embodiments disclosed above, it should be noted that the reduced treatment region allows the therapeutic intervention to be focused on the desired regions of the diseased tissue, for example a tumor, and spares the healthy tissue surrounding the diseased tissue from the undesired side-effects or consequences of the intervention.

At step 140, the flow control element may be removed from the patient after the therapeutic agent has been delivered. Alternatively, the flow control element may be implanted within the patient for an extended period of time to prevent disease dissemination and/or to confine the therapeutic effect of the therapeutic agent.

The following example illustrates one exemplary implementation of the present devices, systems and methods for treating locoregional advanced lung cancer using radiation therapy. The example should not be construed as limiting.

### EXAMPLE 1

A 51 year old man was diagnosed with a 7.8×11 cm large tumor of the right lower lobe of the lung using a CT scan. Due to the lymphonodular enlargement a mediastinoscopy was performed. The histological examination of the lymph nodes (precarinal, bifurcation and pretracheal) revealed cells of a low differentiated adenocarcinoma of the lung. After completion of the staging (MRT of the head, sonography of the abdomen) the patient was diagnosed with locoregional advanced lung cancer of the right lower lobe with TNM classification cT2b, cN2(Medias 4/16) cM0. After interdisciplinary discussion of these findings indication for curative radiation therapy was announced.

Three cycles of chemotherapy with cisplatin/paclitaxel were performed. Thereafter, curative radiation therapy was planned.

The main tumor was located in the periphery of the right lower lobe. In addition to that the mediastinal lymph nodes had to be integrated into the therapy plan, which made the radiation field very large. In order to achieve a curative therapy with protection for the surrounding tissue especially the lung, primaries, and mediastinal lymph nodes had to be brought together. Flow control elements configured as endobronchial valves were implanted to the lung segment; atelectasis of the lung segments was achieved. Due to the shrinking of the atelectatic tissue an approximation of the primarius and the mediastinum was created and therefore the radiation field was decreased.

Specifically, endobronchial valves in the Ostium of B6, B8, B9 (4 mm), and B10 (5 mm) were used to achieve flow control of the right lower lobe of the lung. Atelectasis developed within hours and the approximation of primarius and mediastinal was generated.

Radiation of the larger tumor region was performed with an iso-centric 3-field technique with 15MV-photons. A dose of 44Gy was applied in factions of 5×2Gy per week. Simultaneously chemotherapy with cisplatin (50 mg/m²) and navelbine (20 mg/m²) one treatment day 1 and day 8 was given. Then the radiation of the macroscopic tumor region was added (fractions of 5×2Gy until a complete dose of 64/71 Gy) and combined with chemotherapy with cisplatin (40 mg/m², day 1) and navelbine (15 mg/m², day 1 and 8).

After the completion of radiation, a bronchoscopy was performed. The valves were removed without any complication. No significant secretion could be detected distal of the valves. The segments of the right lower lobe were shortly distended with air in order to achieve a complete re-expansion.

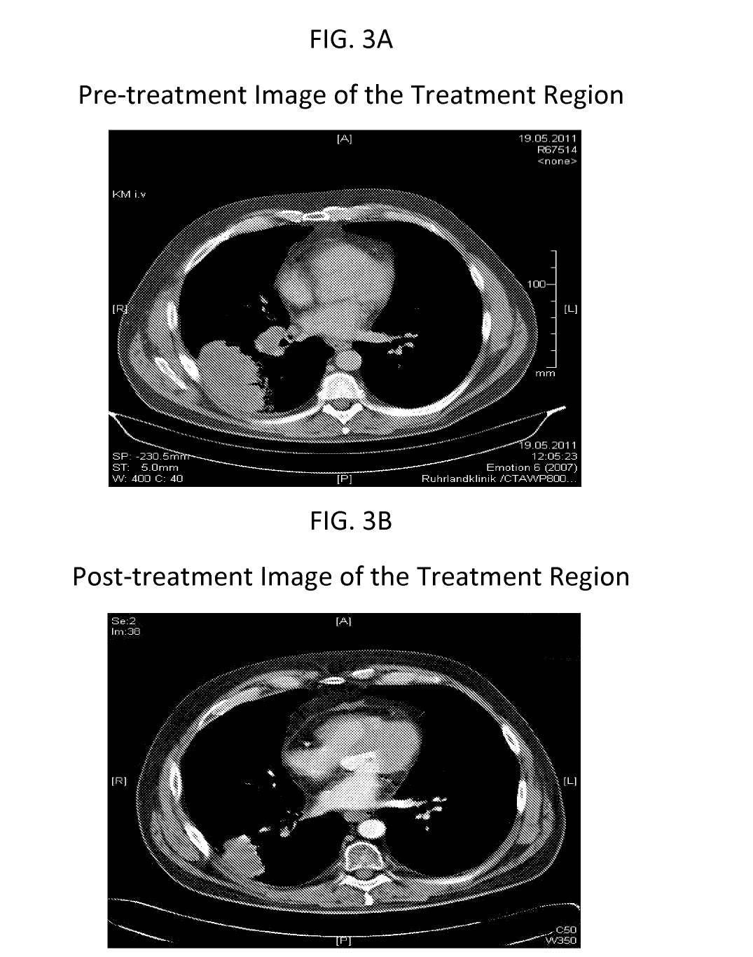

Thereafter the patient described a reduction of his shortness of breath. Pre-treatment and post-treatment x-ray images as shown in FIGS. 3A and 3B indicate a decrease in the size of the tumors post-treatment. Furthermore, pneumothorax and the former atelectasis were no longer detectable post-treatment.

In cases with peripheral primarius and mediastinal lymph node metastasis a curative intended radiation therapy can be difficult. Induction of atelectasis by implantation of flow control elements such as endobronchial valves can reduce the radiation field and optimize this therapy by creating a higher protection of the surrounding tissue.

Additionally and optionally, it is contemplated that prior to the delivery of the therapeutic agent, presence, absence, or degree of collateral ventilation within lung segments can be determined. Normally, the lung segment and its surrounding fibrous septum are intact units. In some patients, however, the fibrous septum separating the lobes or segments may be perforate or broken, thus allowing air flow between the segments, referred to as “collateral ventilation.”

Employing the present devices, systems, and methods on a treatment region within a lung region where collateral ventilation is present may require additional consideration and/or modification since the degree of desired atelectasis may not be achieved by using the flow control element due to collateral ventilation.

Some methods and devices for localized diagnosis and functional testing to identify specific areas of collateral ventilation and/or other disease parameters within the lung are disclosed in co-pending and commonly owned U.S. Published Patent Applications 2007/0142,742, 2008/0249,503 and 2008/0200,797, which are incorporated herein by reference in their entirety. These applications discuss the measurement of collateral ventilation at the lobar and segmental levels in patients with emphysema. The measurement of collateral ventilation is done in a minimally invasive manner by occluding the airway and determining the change in pressure and/or measuring the composition of the gas within the lung compartment. The measurements may then be followed by an appropriate treatment to effect lung volume reduction and the therapeutic agent delivery thereafter. Additionally, localized diagnosis and functional testing by using a physiological testing unit of a pulmonary diagnostic system as exemplarily described in the co-pending application U.S. Ser. No. 10/241,733 may be used to determine one or more physiological characteristics of the lung to determine the suitability of the patient for volumetric reduction and/or further treatment using one or more of therapeutic agents. Furthermore, and a treatment unit connected to the pulmonary diagnostic system may control the delivery of the therapeutic agent based on the at least in part the measurement of the pulmonary diagnostic system.

Although various embodiments described above disclose using one or more flow control elements to achieve volumetric reduction of the treatment region, it is contemplated that other volumetric reduction techniques may be used in conjunction of or instead of the flow control elements. For example, an endobronchial lung volume reduction catheter, vacuum, or the like may be used to achieve volumetric reduction.

Present disclosure also provide one or more kits for use in practicing the one or more methods described herein, where the kits typically include one or more of flow control elements. Kits may also include one or more delivery catheters, loading devices, connectors, or the like. In one embodiment, one or more therapeutic agents could also be included in the kit. In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method of treating a lung region, comprising:
delivering one or more flow control elements to a lung region;
deploying the flow control elements to at least partially collapse the lung region; and
delivering at least one therapeutic agent to the collapsed lung region, wherein the effect of the therapeutic agent is at least partially contained within the collapsed lung region.

2. The method of claim 1, further comprising determining the presence or absence of collateral ventilation in the lung region.

3. The method of claim 1, wherein the flow control elements are endobronchial valves.

4. The method of claim 1, wherein the therapeutic agent is a radioactive agent or radioactive energy.

5. The method of claim 4, wherein the delivering is accomplished by irradiating the collapsed lung region with radioactive energy.

6. The method of claim 4, wherein the delivering is accomplished by placing the radioactive agent in the collapsed lung region.

7. The method of claim 1, further comprising removing the flow control elements after delivering at least one therapeutic agent to the collapsed lung region.

8. A method of treating a body region, comprising:
determining a treatment region;
reducing the volume of the treatment region to create a target region; and
delivering at least one therapeutic agent to the target region;
wherein reducing the volume of the treatment region is achieved by deploying one or more flow control elements to reduce fluid flow into the treatment region.

9. The method of claim 8, wherein the therapeutic agent is a radioactive agent or radioactive energy.

10. The method of claim 9, wherein the delivering is accomplished by irradiating the target region with radioactive energy.

11. The method of claim 9, wherein the delivering is accomplished by placing the radioactive agent in the target region.

12. The method of claim 8, wherein the flow control elements are one-way valves.

13. The method of claim 8, wherein the treatment region is a lung region.

14. The method of claim 13, further comprising determining the presence or absence of collateral ventilation in the lung region.

15. The method of claim 8, wherein the reducing the volume of the treatment region is achieved by at least partially collapsing the lung region.

16. The method of claim 8, further comprising removing the flow control elements after delivering at least one therapeutic agent to the collapsed lung region.
